(19) United States
(12) Patent Application Publication
(10) Pub. No.: US 2016/0143603 A1
(43) Pub. Date: May 26, 2016

(54) Imaging with Ramping

(71) Applicant: General Electric Company, Schenectady, NY (US)

(72) Inventor: David Michael Hoffman, Waukesha, WI (US)

(73) Assignee: General Electric Company, Schenectady, NY (US)

(21) Appl. No.: 14/549,250

(22) Filed: Nov. 20, 2014

(51) Int. Cl. A61B 6/00; A61B 6/03

(52) U.S. Cl. CPC A61B 6/482; A61B 6/032; A61B 6/545

(57) ABSTRACT

An apparatus for capturing images is described herein. The apparatus may include a generator to generate energy to be emitted at an imaging device. The apparatus may also include a controller, at least partially including hardware logic, to direct the generator to ramp energy emitted at the imaging device from a first energy level to a second energy level in a ramping waveform.

### BACKGROUND OF THE INVENTION

The subject matter disclosed herein relates generally to an apparatus and method for diagnostic medical imaging. In these variations of diagnostic imaging systems, multiple detectors or detector heads may be used to capture an image of a subject, or to scan a region of interest. For example, the detectors may be positioned near the subject to acquire imaging data, which is used to generate an image of the subject. For example, CT systems may make use of dual energies wherein images are captured at two different kilovolt (kV) energy levels within a computed tomography view. A dual energy use within a CT view is sometimes called “fast Kv.” Gathering imaging information for two energies may enable energy discrimination scanning Energy discrimination scanning may include the subtraction of the image data gathered at a first energy level from the image data gathered at a second energy level. An energy difference may provide greater clarity and discrimination in the resulting data set and its accompanying images.

### BRIEF DESCRIPTION OF THE INVENTION

An embodiment relates to an apparatus for capturing image data. An apparatus for medical imaging may include a generator and an X-ray tube to generate energy to be emitted at an imaging device. The apparatus may further include a controller, at least partially comprising hardware logic, to direct the generator to ramp energy emitted at the imaging device from an x-ray tube from a first energy level to a second energy level in a ramping waveform.

Another embodiment relates to a method of acquiring imaging data. This embodiment of the method may include generating energy, with a generator and an X-ray tube, to be emitted at an imaging device. This embodiment of a method also involves including directing the generator, with a controller, at least partially including hardware logic, to ramp energy emitted at the imaging device from a first energy level to a second energy level in a ramping waveform.

Still another embodiment relates to a system of obtaining imaging data. This embodiment of a system may include a detector of an imaging device, an imaging emitter of the imaging device such as an X-ray tube, and a generator to generate energy to be emitted at the imaging device. This embodiment of a system may also include a controller, at least partially including hardware logic, to direct the generator to ramp energy emitted at the imaging device by the imaging emitter from a first energy level to a second energy level in a ramping waveform.

### BRIEF DESCRIPTION OF THE DRAWINGS

The present techniques will become more fully understood from the following detailed description, taken in conjunction with the accompanying drawings, wherein like reference numerals refer to like parts, in which:

FIG. 1 illustrates a diagram of a medical imaging system;

FIG. 2 illustrates a simplified block diagram of a system for obtaining imaging data;

FIG. 3 illustrates a simplified process flow diagram of a method for obtaining imaging data;

FIG. 4 illustrates a diagram of a number of waveforms including a number of ramping waveforms; and

FIG. 5 illustrates an exemplary graph to illustrate the k-edge absorption of various materials at various energies.

### DETAILED DESCRIPTION OF THE INVENTION

In the following detailed description, reference is made to the accompanying drawings that form a part hereof, and in which is shown by way of illustration of specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the embodiments described herein.

As used herein, the terms “system,” “unit,” or “module” may include a hardware and/or software system that operates to perform one or more functions. For example, a module, unit, or system may include a computer processor, controller, or other logic-based device that performs operations based on instructions stored on a tangible and non-transitory computer readable storage medium, such as a computer memory. Alternatively, a module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

Various embodiments provide a new methodology for improved energy discrimination scanning with existing medical imaging systems, such as CT systems and today's detector. For example, today's CT systems make use of dual kilovolt (kV) levels, also sometimes known as “fast kV.” In fast kV, dual energy levels are used to scan an object to produce an image having both energy levels displayed within a single view in order to provide two sets of image data, one at each energy level.

In the techniques described herein, a beam with an energy peak, or a peak kilovoltage (kVp) that is ramped is used to capture image data during the ramped voltage. Specifically, within a single CT view from one kVp to another kVp image data is captured during a ramped voltage emission. Ramping, as referred to herein, is a continuous increase or decrease of kV levels. For example, these ramped energies could go from 80 kVp to 104 kVp. The ramp and its values could however cover any range of kVp.

With a ramped kVp, each view could then be broken into a number of subviews, in some examples, through the use of distinct integration periods measuring a charge generated in response to a detector detecting the emitted energy. In some examples the number of subviews or distinct integration periods could include 5-7 integration periods per view. In this example, it may also be necessary to increase the sampling rate by a number commensurate with the number of integration periods. For example, 1000 views per CT rotation having a 0.2 second scanning with 5 sub integrations per view may indicate that the data acquisition system receiving the image information will operate at 25 KHz.

In other embodiments, a subsequent view and accompanying scans could also ramp the kVp in the opposite direction, e.g. high to low and low to high. Depending on the sampling speed desired, various components may need to be upgraded to accommodate the ramping's increase in the ability to sample a much larger number of energies. One example of this could include upgrading the scintillator from a gemstone scintillator to something with a faster time constant. Further, it should be understood that the exact ramping rate need not always be linear so long as it is a continuous function. The actual Kv waveform within the view could follow any waveform shape no matter how complex the waveform selected may be. In some examples, the Kv waveform could include a sine wave or any other continuous or stepping function. Accordingly, an energy ramp may follow an exponential ramping waveform, a logarithmic waveform, or any other continuous functions.

In some examples, the data obtained from these ramped samples may also have any energy overlap between samples and subsamples. However, the degree of subsample, or subview, separation may be improved with variable X-ray filtering per subsample and through the use of k-edge properties to improve imaging. A k-edge, as referred to herein, may also be thought of as an x-ray absorption edge of various elements. For example, iodine has a unique edge due to its atomic number. In medical imaging, if iodine resides inside a body being scanned, ramping energies in the scans will cross a point where the x-ray absorption changes dramatically. This point can be visualized on a graph or through statistical analysis and is called a k-edge. This property of elements may be useful when harnessed by the presently disclosed system for the purpose of diagnostic imaging. Specifically, leveraging the k-edge now detectable thanks to the disclosed ramping functions utilized in imaging enables an improvement in material identification, and therefore overall medical image quality. Another benefit of this improved methodology is that as each element has its own unique k-edge, each element may now be used as an effective contrast material. As the effect of contrast material on a body and an image is often a difficult challenge to keep safe and effective, the broader range of options will allow more flexibility in target contrast material selection. It also may allow for the use of specific target contrast materials to highlight tumors, cancers, and other similar masses and anatomical detail.

The presently disclosed embodiments enable medical scanning that may make use of several ramping energy functions to specifically target the k-edge absorptions ranges for a material. Further, the techniques described herein include ramping energy emitted for a number of subviews enabling several k-edges to be reviewed, if sufficient image samples are taken.

It should be noted that although the various embodiments are described in connection with a particular CT imaging system, such as ramping within fast kV, the various embodiments may be implemented in connection with other imaging systems. Additionally, the imaging system may be used to image different objects, including objects other than people.

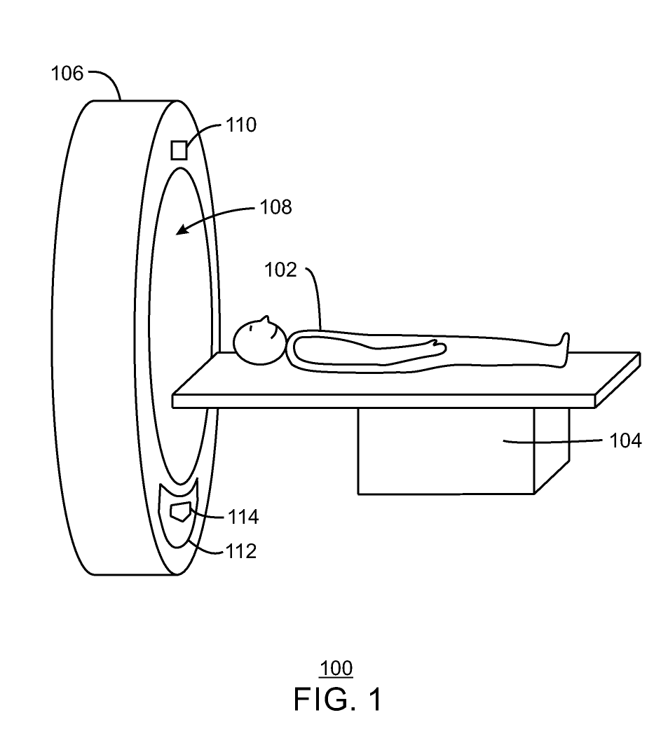

FIG. 1 illustrates a diagram of a medical imaging system. In the system 100, a subject 102 can be a human patient in one embodiment. It should be noted that the subject 102 does not have to be human. In embodiments, the subject is some other living creature or inanimate object. As illustrated in FIG. 1, the subject 102 can be placed on a pallet bed 104 that can move a subject horizontally for locating the subject 102 within a gantry 106. The gantry 106 is shown as circular in one embodiment. In other embodiments the gantry 106 may be of any shape such as square, oval, “C” shape, a hexagonal shape, and the like. In one embodiment, the subject 102 may be located by the pallet bed 104 in the most advantageous imaging position within a bore 108 of a gantry 106.

An imaging emitter such as an x-ray tube 110 is shown on the gantry above the subject 102. While the imaging emitter 110 is shown on the gantry 106 in a particular position, this does not exclude additional locations for the imaging emitter 110 such as within the walls of the gantry 106 or within the bore 108 of the gantry 106. The imaging emitter 110 may emit a beam of x-rays in one example. However, this does not exclude any other type of electromagnetic radiation, or, for that matter, any other imaging emitter which emits in order to facilitate imaging. A detector 112 is also shown on the gantry 106. The detector 112 may be used to detect any x-rays or other signal sent by the imaging emitter 110. Similar to the imaging emitter 110, the detector 112 may be located or affixed in a variety of configurations as needed by the attributes of a particular imaging system 100.

A data acquisition system 114 (DAS) is shown within a detector 112 on the gantry 106. Again this positioning is only one embodiment and the DAS 114 may be located elsewhere. In one embodiment, the DAS 114 integrates a charge that changes based on a signal the detector 112 may be receiving in order to change the charge form analog data to digital data for use by a processor or computer. The DAS 114 may be associated with, or include, a controller (not shown) configured to ramp energy emissions at the imaging emitter 110. This controller could also alternatively be located within the generator or anywhere else within the CT gantry. As discussed above and in more detail below, ramping of energy emissions may provide beneficial indications in a captured image. As stated above, FIG. 1 is a simplified diagram. Other components may be present or even necessary in a functioning imaging system, however for simplicity these components are not shown.

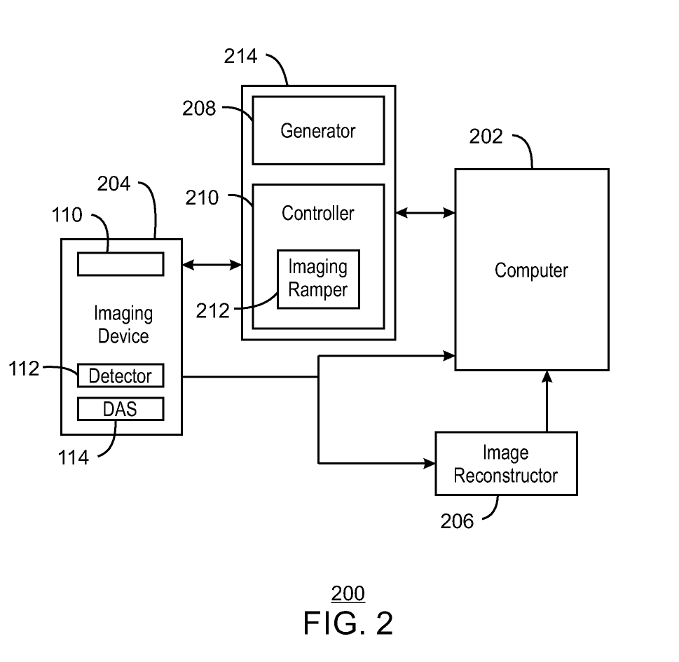

FIG. 2 illustrates a simplified block diagram of a system 200 for obtaining imaging data. A computer 202 is shown as part of this system and may be any type of computer or workstation used for imaging or image processing. Further, while the computer 202 is shown separately from the other items, each item may be a part of the other, in different locations (i.e. cloud storage or remote locations). For example, the computer may be inseparably physically a part of an imaging device 204. The imaging device 204 may be an imaging system, such as the imaging system 100 of FIG. 1 used to image items or subjects 102. The simplified diagram used for FIG. 2 does not include a gantry 106 or a pallet bed 104 or many other components of an imaging system for simplicity. The imaging emitter 110 and detector 112 are shown as included in this figure. While in both FIG. 1 and FIG. 2 the imaging emitter 110 and the detector 112 are shown on opposing sides of the gantry 106 or imaging device 204, this configuration and orientation is merely exemplary and these components may be placed in any configuration necessary to perform their respective functions.

In embodiments, X-rays leave an imaging emitter 110 and may go through a subject 102. In some embodiments, a collimator is placed in front of the detector 112 to reduce scatter radiation coming from off angles of an emitted beam. Below a collimator, some embodiments of a detector 112 may include a scintillator to absorb the x-rays, or other radiation coming directly through the collimator, and to convert this radiation to visible light. This light may be received by a photodiode, may produce a current when light is shined on it. This diode and current configuration may be connected to a set of electronics, such as the DAS 114 of FIG. 1, configured to integrate the current or charge over a set amount of time. In other words, the DAS 114 may collect information such as a charge or number of electrons detected over a period of time as they are received by the photodiode. The DAS 114 may integrate this charge for the view period (ex. 1/1000 of a rotation), thereby changing the amount of charge from analog (current) to digital (a value). All of this imaging information may then be sent to a computer 202 and may also be sent to an image reconstructor 206 to aid in generation of an image from the raw detected data.

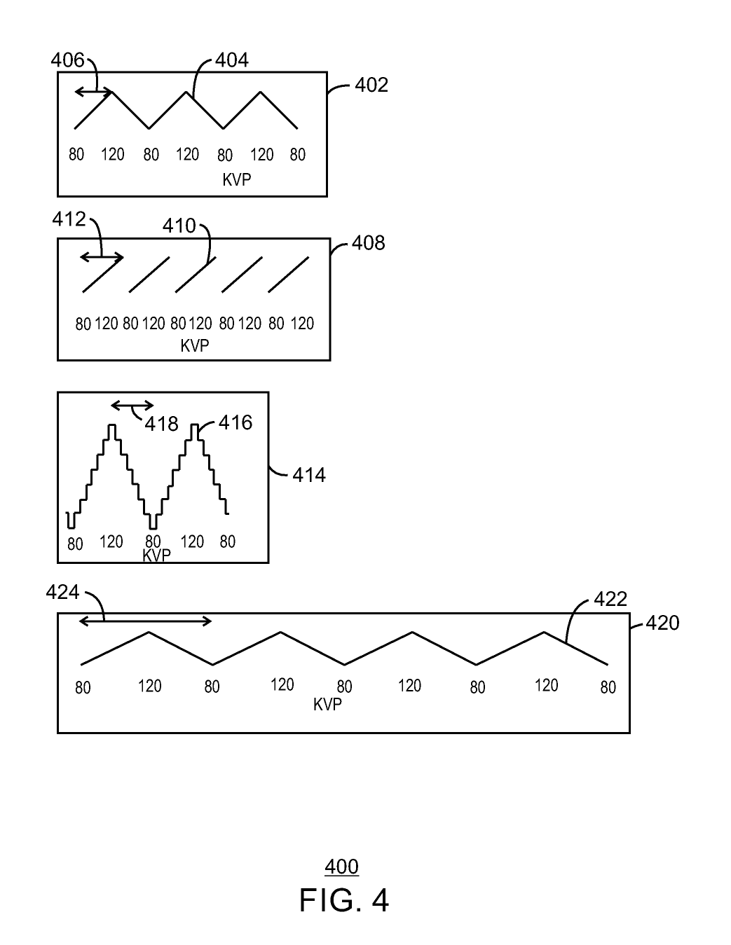

A generator 208 may be configured to generate energy which will eventually be passed to an emitter 110 and emitted at an imaging device 204. A controller 210 or controller apparatus is also herein disclosed as a device which enables the ramping of energies, or kVp in an imaging scan. The controller 210 may include logic, at least partially including hardware logic, such as an integrated circuit, firmware, software to be executed by a processing device, or electronic circuit logic. In some cases, the controller 210 may also provide control and coordination for the imaging process. For example, the controller 210 may also direct any rotation of the emitter 110 and detector 112 that may be needed in imaging. Rotation direction may include dividing a section to be imaged into a number of rotational angles. The controller 210 may be configured to manage this division into rotational angles and may also trigger the rotation of components along those rotational angles. The controller 210 may also be configured to provide access to power in varying quantities to an imaging emitter 110 and may also be configured to provide similar access to power for other components. An imaging ramper 212 or imaging ramping module may also be contained within the controller 210. The imaging ramper 212 may contain a specific pattern or description of a ramped waveform that a controller may use to variously provide power to the imagining emitter 110. The imaging ramper 212 may vary the ramped waveform in response to commands from the computer 202. The imaging ramper 212 may modify the waveform to take a different pattern or form, variations of which are shown in more detail in FIG. 4.

In some embodiments, the imaging ramper 212 enables the controller 210 to energize the imaging emitter 110 at the appropriate level in an energy ramp such that a k-edge can be detected through the multiple subviews, energies, and detection methods enabled by the disclosed components. In some embodiments each of these components are all combined within an apparatus 214 for generating energy with the generator, controlling the imaging device 204 with the controller 210, and providing a ramping waveform with the imaging ramper 212.

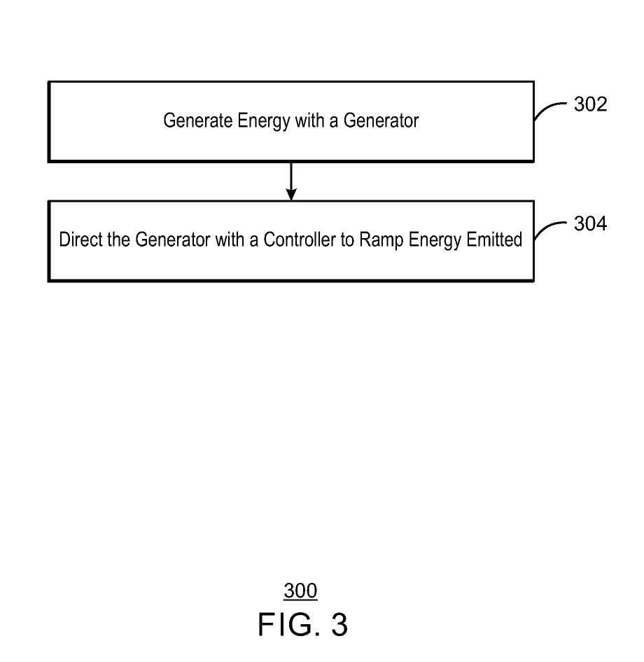

FIG. 3 illustrates a simplified process flow diagram of a method 300 for obtaining imaging data. In some embodiments, this exemplary method may be used in conjunction with the systems and apparatuses shown in FIGS. 1 and 2. Generally, FIG. 3 illustrates how some method embodiments acquire the imaging data.

At block 302, the method includes generating energy, with a generator, to be emitted at an imaging device. This generation of energy may be later transmitted in beam form from an imaging emitter.

At block 304, the method includes directing the generator, with a controller, at least partially comprising hardware logic, to ramp energy emitted at the imaging device from a first energy level to a second energy level in a ramping waveform. This ramping may provide additional clarity in generated images.

In some embodiments, the imaging emitter may emit x-rays at a variety of energies or kVp's. The beam itself may be an x-ray beam but may also be any other emittable item suitable for medical imaging.

In other examples, the method may include generating an energy level of the beam for each of a first plurality of subviews. In these scenarios, the method may include receiving imaging data with a detector for the first set of subviews within the first view. In some embodiments, these subviews are not the same as the first view and second view elsewhere discussed as the subviews may be only regarding a ramping time period of emission and detection, while the first and second view may refer to angular positions at which a beam is emitted around a subject, such as the subject 102 of FIG. 1. The subviews may also refer to an integration of the detected data within a ramping time period associated with a subview. For example, a ramping time period may be 1 milli-second wherein energy is emitted continuously from 80 kVp to 120 kVP. Imaging data received and integrated over the 1 milli-second period of the ramping cycle may be one or more subviews for any given angular position. In some embodiments the first and second view may also have a time element.

The example method may further include energizing the imaging emitter to project a beam at a second view based on the ramping waveform such that the detector receives imaging data for the same beam projected energy levels in both the first view and the second view. In this scenario, the imaging data captured is for the same energy values across every view taken of the subject. For example, if imaging ramping has allowed a detected density to be measured at one energy of beam emission in one view, that same beam energy should be replicated at each view in order to be able to reconstruct full sets of imaging data. In some embodiments, the ramping generates a nearly limitless number of data points, or beam kVps from which to choose for imaging data. Accordingly, data discrimination possibilities also grow in some embodiments of the presently disclosed embodiment.

In some cases, the method may include receiving imaging data with a detector for a second plurality of subviews within the second view. Although in some embodiments the method may be executed in this shown order, the disclosed embodiments are not so limited. Accordingly additional steps may be added or removed while still in the scope of some of disclosed embodiments.

FIG. 4 illustrates a diagram of a number of waveforms 400 including a number of ramping waveforms. These waveforms that may be used are many, with some illustrated in FIGS. 1, 2, and 3 for example to affect a change in the energy level of an emitted beam. A sawtooth kV section 402 is shown and includes a sawtooth ramping kVp 404. The sawtooth ramping kVp 404 is shown over a single view 406. In addition to providing the benefits of a ramping kVp discussed above and in more detail below, the sawtooth ramping kVp also does not need to reset it's voltage between views and instead may increment or decrement kVp based on its ending energy level in a previous view.

A unidirectional kV section 408 is shown and includes a unidirectional ramping kVp 410. The unidirectional ramping kVp 410 is shown over a single view 412. In addition to providing the benefits of a ramping kVp discussed elsewhere, the unidirectional ramping kVp also will not require additional manipulation of data in an image reconstruction phase as each data set is already aligned to the pervious and upcoming views.

A multistep kV section 414 is shown and includes a multistep ramping kVp 416. The multistep ramping kVp 416 is shown over a single view 418. In addition to providing the benefits of a ramping kVp discussed elsewhere, the multistep ramping kVp 416 also may provide clearer delineation between various subviews imaged.

A multipass ramping kV section 420 is shown and includes a multipass ramping kVp 422. The multipass ramping kVp 422 is shown over multiple views 424. For example here the multipass ramping kVp 422 is shown here ramping an emitted beam energy level up and down within one view 424. In addition to providing the benefits of a ramping kVp discussed elsewhere, the multipass ramping kVp 422 also may provide increased accuracy and discrimination of imaging as two sets of subviews, or two views at various energy states may be generated for a single view and the detected values averaged by the image reconstructor. Further, the Kv waveform shape within a view or over one or more views can be any continuous or any stepped waveform function of any shape and any complexity Including a sine wave or other various continuous or step functions.

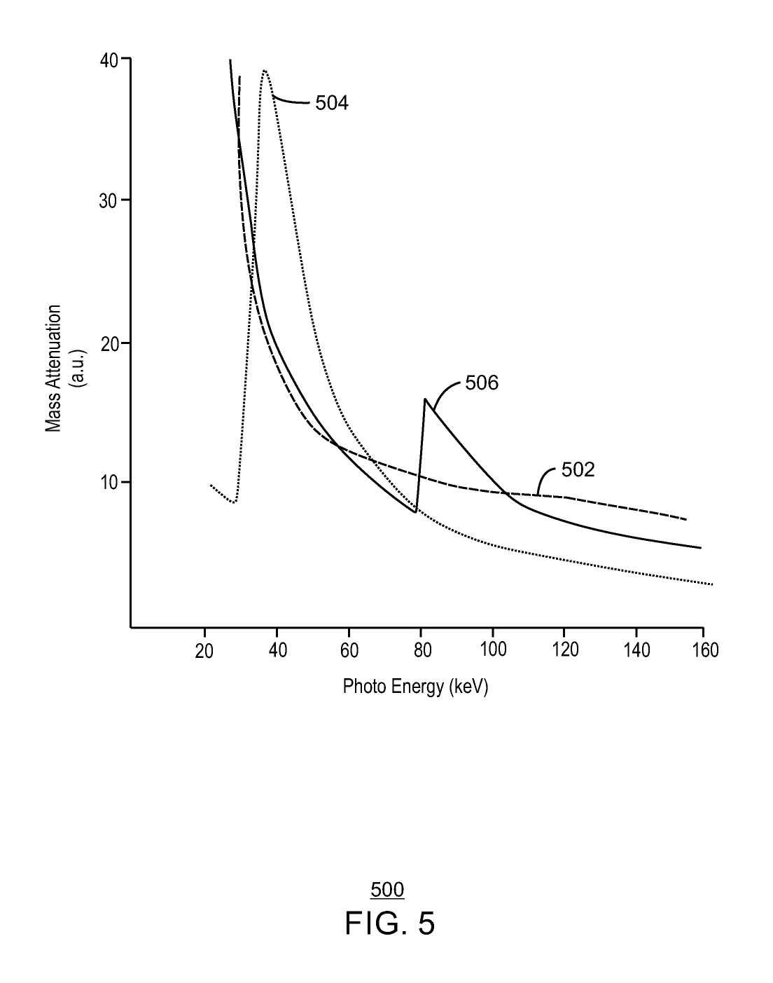

FIG. 5 illustrates an exemplary graph 500 to illustrate the k-edge absorption of various materials at various energies. As discussed above, the k-edge absorption detection capabilities enabled by the presently disclosed embodiments provide increased distinctiveness to detected images. To illustrate an absorption pattern that indicates an elements k-edge, this exemplary graph 500 contains approximated data of x-ray absorption displayed by various elements or components shown by each component's normalized mass attenuation coefficients at various photon energies. For reference, it is these photon energies which may be varied, in some embodiments, by the controller 210 though a beam energy level in a beam emitted by an imaging emitter 110 and detected in a set of subviews by a detector 112.

Line 502 illustrates the normalized mass attenuation by calcium carbonate at varying photon energies. As this is a molecule with more than one element and low atomic number elements, it has a variety of attenuations, and less distinctive lower energy k-edges not seen in this plot. It may be noted as well that k-edges for atoms in molecules are just as easy to image. In contrast, line 504 represents elemental iodine and accordingly displays a large k-edge at a photon energy of approximately 40 keV. This is seen in the relative spike in the normalized mass attenuation of the iodine 504 at this value. Similarly, line 506 illustrates the normalized mass attenuation for elemental gold. As seen on the graph 500, there is a spike in mass attenuation for gold 506 at approximately 80 keV, a k-edge for gold. As these k-edges enable unique identification of specific elements to be used as target marking materials, they are very valuable to imaging techniques. Currently, the ramping systems and method embodiments proposed enable the use of k-edge properties in imaging. Further, more than one k-edge may be detected as ramping enables an almost limitless variability in specific energies emitted and number of subviews detected.

While the detailed drawings and specific examples given describe particular embodiments, they serve the purpose of illustration only. The systems and methods shown and described are not limited to the precise details and conditions provided herein. Rather, any number of substitutions, modifications, changes, and/or omissions may be made in the design, operating conditions, and arrangements of the embodiments described herein without departing from the spirit of the present techniques as expressed in the appended claims.

This written description uses examples to disclose the techniques described herein, including the best mode, and also to enable any person skilled in the art to practice the techniques described herein, including making and using any devices or systems and performing any incorporated methods. The patentable scope of the techniques described herein are defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An apparatus for medical imaging, comprising:
a generator to generate energy to be emitted at an imaging device;
a controller, at least partially comprising hardware logic, to direct the generator to ramp energy emitted at the imaging device from a first energy level to a second energy level in a ramping waveform.

2. The apparatus of claim 1, wherein the controller is to adjust the ramping waveform generating a first plurality of subviews for a first view and a second plurality of subviews for a second view such that the imaging data received reveals a k-edge of a marking material.

3. The apparatus of claim 1, wherein the ramping waveform generates a continuous energy emission at the imaging device between the first energy level and the second energy level.

4. The apparatus of claim 1, wherein the controller is further to direct the imaging device to:
rotate an imaging emitter and a detector of the imaging device around an object to be imaged;
project a beam at a first view based on the ramping waveform generating a first plurality of subviews within the first view; and
project a beam at a second view based on the ramping waveform generating a second plurality of subviews within the second view, wherein the first view and the second view represent different rotational angles around the object to be imaged.

5. The apparatus of claim 1, wherein the ramping waveform comprises:
a multipass ramping waveform that ramps an emitted beam energy level up and down twice in one view;
a sawtooth ramp waveform wherein the ramping may increment or decrement kilovoltage peak starting at an ending energy level in a previous view;
a unidirectional ramp waveform wherein the ramping resets at a kilovoltage peak value in every view before ramping data either up or down;
or any combination thereof.

6. The apparatus of claim 1, wherein the ramping waveform is a continuous function wherein an emitted energy level of the waveform comprises:
a linear energy level emission;
an exponential energy level emission;
a logarithmic energy level emission; or
any combination thereof

7. The apparatus of claim 1, wherein the ramping waveform only affects the energy of an emitted beam such that the beam is only projected between a maximum energy level and a minimum energy level.

8. An method for medical imaging, comprising:
generating energy, with a generator, to be emitted at an imaging device;
directing the generator, with a controller, at least partially comprising hardware logic, to ramp energy emitted at the imaging device from a first energy level to a second energy level in a ramping waveform.

9. The method of claim 8, further comprising adjusting the ramping waveform to generate a first plurality of subviews and a second plurality of subviews such that the imaging data received reveals a k-edge of a marking material.

10. The method of claim 8, further comprising generating, via the ramping waveform, a continuous energy emission at the imaging device between the first energy level and the second energy level.

11. The method of claim 8, further comprising:
rotating an imaging emitter and a detector of the imaging device around an object to be imaged;
projecting a beam at a first view based on the ramping waveform generating a first plurality of subviews within the first view; and
projecting a beam at a second view based on the ramping waveform generating a second plurality of subviews within the second view, wherein the first view and the second view represent different rotational angles around the object to be imaged.

12. The method of claim 8, wherein the ramping waveform comprises:
a multipass ramping waveform wherein that ramps an emitted beam energy level up and down twice in one view;
a sawtooth ramp waveform wherein the ramping may increment or decrement kilovoltage peak starting at an ending energy level in a previous view;
a unidirectional ramp waveform wherein the ramping resets at a kilovoltage peak value in every view before ramping data either up or down;
or any combination thereof.

13. The method of claim 8, wherein the ramping waveform is a continuous function wherein an emitted energy level of the waveform comprises:
a linear energy level emission;
an exponential energy level emission;
a logarithmic energy level emission; or
any combination thereof.

14. The method of claim 8, wherein the ramping waveform only affects the energy of an emitted beam such that the beam is only projected between a maximum energy level and a minimum energy level.

15. A system to acquire imaging data, comprising:
a detector of an imaging device;
an imaging emitter of the imaging device;
a generator to generate energy to be emitted at the imaging device; and
a controller, at least partially comprising hardware logic, to direct the generator to ramp energy emitted at the imaging device by the imaging emitter from a first energy level to a second energy level in a ramping waveform.

16. The system of claim 15, wherein the controller is to adjust the ramping waveform generating a first plurality of subviews, and a second plurality of subviews such that the imaging data received reveals a k-edge of a marking material.

17. The system of claim 15, wherein the ramping waveform generates a continuous energy emission at the imaging device between the first energy level and the second energy level.

18. The system of claim 15, wherein the controller is further to direct the imaging device to:
rotate an imaging emitter and a detector of the imaging device around an object to be imaged;
project a beam at a first view based on the ramping waveform generating a first plurality of subviews within the first view; and
project a beam at a second view based on the ramping waveform generating a second plurality of subviews within the second view, wherein the first view and the second view represent different rotational angles around the object to be imaged.

19. The system of claim 15, wherein the ramping waveform comprises:
a multipass ramping waveform wherein that ramps an emitted beam energy level up and down twice in one view;
a sawtooth ramp waveform wherein the ramping may increment or decrement kilovoltage peak starting at an ending energy level in a previous view;
a unidirectional ramp waveform wherein the ramping resets at a kilovoltage peak value in every view before ramping data either up or down;
or any combination thereof.

20. The system of claim 15, wherein the ramping waveform is a continuous function wherein an emitted energy level of the waveform comprises:
a linear energy level emission;
an exponential energy level emission;
a logarithmic energy level emission; or
any combination thereof
